(19) United States
(12) Patent Application Publication
(10) Pub. No.: US 2013/0041285 A1
(43) Pub. Date: Feb. 14, 2013

(54) BIOPSY EXTRACTION UNIT

(71) Applicants: Dan Akerfeldt, Uppsala (SE); Gunnar Aström, Uppsala (SE)

(72) Inventors: Dan Akerfeldt, Uppsala (SE); Gunnar Aström, Uppsala (SE)

(73) Assignee: APRIOMED AB

(21) Appl. No.: 13/642,979

(22) PCT Filed: Apr. 18, 2011
(86) PCT No.: PCT/SE2011/050475

§ 371 Date: Oct. 23, 2012

(30) Foreign Application Priority Data
Apr. 27, 2010 (SE) 1050419-9

(51) Int. Cl. A61B 10/02

(52) U.S. Cl. 600/567

(57) ABSTRACT

Biopsy extraction unit for use in relation to a biopsy device provided with an outer cannula. The biopsy extraction unit is provided with a unit handle, an intermediate part and an elongated sample rod, the rod being offset and essentially parallel to the longitudinal central axis of the biopsy extraction unit, wherein the sample rod is adapted to essentially be positioned along an inner wall of the outer cannula when inserted into said biopsy device.

### FIELD OF THE INVENTION

The present invention relates to biopsy extraction unit, and a method in relation thereto, according to the preambles of the independent claims.

### BACKGROUND OF THE INVENTION

In the prior art, as exemplified by U.S. Pat. No. 6,063,037; U.S. Pat. No. 6,755,793; U.S. Pat. No. 7,033,324, and U.S. Pat. No. 6,443,910, an outer cannula used for biopsy is provided with an inner cannula slidable within the outer cannula and used to grasp the tissue when withdrawing the outer cannula.

Often the inner cannula is provided with longitudinal slits in the distal end that cooperates with the inner conical shape of the outer cannula in order to grasp and hold the tissue sample when the entire biopsy needle is withdrawn.

It is sometimes desired to have a biopsy needle with a small outer diameter and when reducing the outer diameter of the biopsy needle the inner needle provided with longitudinal slits often is difficult to manufacture and use due to the small dimensions. E.g. the risk increases that the inner needle will be deformed during use.

The inventor has identified that problem, in particular when using small inner cannulas provided with slits.

Traditional biopsy devices used today has an outer cannula having an outer diameter of 3-4 mm and has a material thickness of approximately 10% of that diameter, i.e. 0.3-0.4 mm. The distal end of such a biopsy device has often an inner conical shape in order to reduce the friction to the tissue sample which facilitates the sample to be pressed into the tube of the biopsy device. Due to the conical shape the inner diameter at the distal end is approximately 15% less then the inner diameter of the rest of the cannula.

As an example, a biopsy device having an outer cannula with an outer diameter of 3 mm will then have an inner diameter of 2.4 mm and an inner diameter at the very distal end being approximately 2.0 mm. This will also be the diameter of the tissue sample. In this case an inner cannula provided with slits would have a material thickness of approximately 0.15 mm to be inserted into the outer cannula without destroying the tissue sample. Furthermore, there is a risk that the distal end of the inner cannula that is provided with the slits would be deformed and the inner cannula must then be discarded after only been used once.

There is an ongoing trend towards biopsy devices having smaller diameters mainly in order to reduce the trauma during the sampling procedure. In order to still be able to take tissue samples having the same size, e.g. diameter, the material thicknesses of both the outer and inner cannulas must be reduced. In addition, the inner conical shape of the outer cannula will be less, which in turn have negative impacts of the inner cannula's ability to firmly hold the tissue sample. This results in that the space for an inner cannula with slits is reduced and that its material thickness will be so thin that it is very difficult to manufacture and to handle it without being deformed.

Thus, an inner cannula provided with slits requires a reduction of the inner diameter at the outer cannula's distal end in order for the inner cannula to work as intended. The inner cannula requires some free space around the tissue sample when it is inserted in order not to expel the sample when the inner cannula is inserted. The diameter reduction results in that the distal end of the inner cannula is forced inwards in the radial direction and locks the tissue sample inside the biopsy device tube. The distal tip of the inner cannula may then be deformed and unusable after only one tissue sampling.

The object of the present invention is to solve the problems described in relation to tissue sampling, and in particular in relation to tissue sampling using biopsy devices having smaller outer diameters.

### SUMMARY OF THE INVENTION

The above-mentioned object is achieved by the present invention according to the independent claims.

Preferred embodiments are set forth in the dependent claims.

Thus, according to present invention the above problem is solved by instead of the inner cannula using a biopsy extraction unit provided with a sample rod that is inserted into the outer cannula such that the sample rod is inserted along the inner wall of the outer cannula, i.e. between the inner wall and the tissue sample, and thereby holding the tissue within the biopsy tube.

The sample rod have a diameter that may be several times larger than the material thickness of a presently used inner cannula and is thereby easier to manufacture and there is no risk that the sample rod will be deformed during use. The biopsy extraction unit may therefore be used several times. In addition, the reduction of the inner diameter of the outer cannula is not required for achieving the tissue sampling.

The sample rod has a very little surface being in contact with the tissue sample when the rod is inserted which result in that the friction between the sample rod and the tissue sample is considerable low.

The tissue sample is held, or locked, within the outer cannula due to the radial forces that the sample rod exerts of the sample tissue that is pressed towards the relatively large inner surface opposite the position of the sample pin. The tissue sample is held in place by the friction of the inner surface of the outer cannula during withdrawal of the biopsy device.

### SHORT DESCRIPTION OF THE APPENDED DRAWINGS

FIG. 1 is a side view of the biopsy extraction unit according to a first embodiment of the present invention.

FIG. 2 is a side view of the biopsy extraction unit according to a second embodiment of the present invention.

FIGS. 3-5 are cross-sectional side views illustrating the use of the biopsy extraction unit in relation to a biopsy device.

FIG. 6 is a cross-sectional view of A-A in FIG. 5 of the biopsy extraction unit in relation to the biopsy device according to one embodiment.

FIG. 7 is a cross-sectional view of A-A in FIG. 5 of the biopsy extraction unit in relation to the biopsy device according to another embodiment.

### DETAILED DESCRIPTION OF PREFERRED EMBODIMENTS OF THE INVENTION

The present invention will now be described with references to the appended drawings.

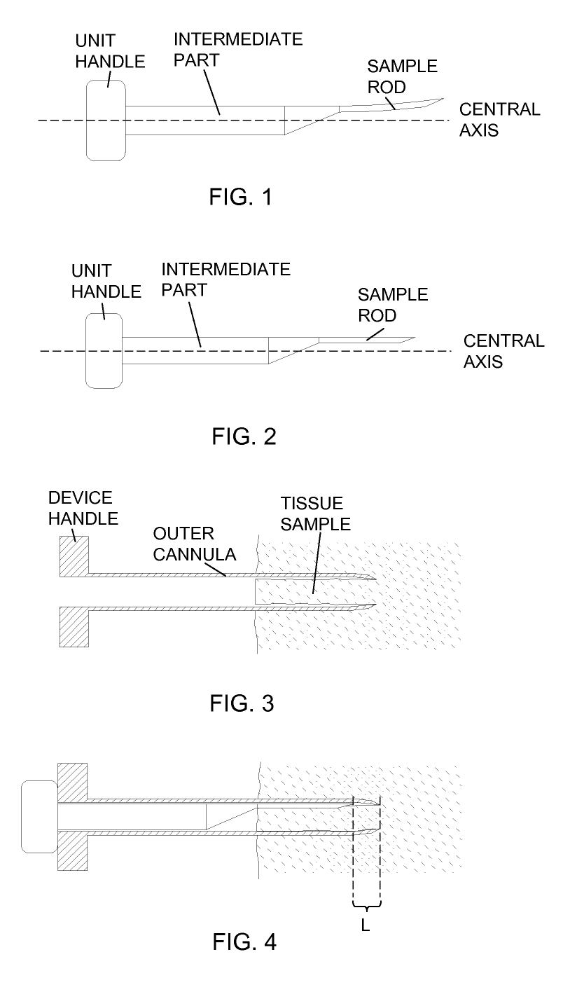

FIG. 1 is a side view of the biopsy extraction unit according to a first embodiment of the present invention.

FIG. 2 is a side view of the biopsy extraction unit according to a second embodiment of the present invention.

The biopsy extraction unit as illustrated in FIGS. 1 and 2 is provided with a unit handle, an intermediate part and an elongated sample rod. The extraction unit is adapted for cooperation with a biopsy device provided with an outer cannula which is illustrated in FIG. 3. The outer cannula is attached to a device handle. The distal end of the outer cannula is sharpened in order to facilitate insertion into the tissue in order to take a tissue sample from a desired location. The intermediate part has preferably a circular cross-section and a cross-sectional shape that fits within the outer cannula such that the extraction unit easily may be inserted into the cannula. The intermediate part has a guiding purpose, i.e. to position the sample rod in relation to the inner surface of the outer cannula such that the sample rod is positioned along and close to the wall.

The sample rod being offset and essentially parallel to the longitudinal central axis of the biopsy extraction unit. The central axis is illustrated by a dashed line in FIGS. 1 and 2.

When using the biopsy extraction unit the sample rod is adapted to essentially be positioned along an inner wall of the outer cannula when fully inserted into the cannula.

The distal tip of the sample rod is sharpened such that the sharpened edge is located furthest away from the central axis in the radial direction. This is clearly illustrated in FIGS. 1 and 2. The reason to sharpen the sample rod in this way is to minimize the influence by the sample rod of the tissue sample when the sample rod is inserted into the outer cannula.

In a first embodiment the distal part of the sample rod is bent outwards with regard to the central axis. This is illustrated by FIG. 1. The sample rod has a certain flexibility permitting it to flex in the radial inward direction when inserted into the outer cannula but still exerting a pressure outwards towards the inner surface. In an initial position, i.e. prior the sample rod is inserted into the outer cannula the most distal part of the sample rod is approximately 50-100% farther away from the central axis in the radial direction compared to the most proximal part of the sample rod, i.e. the part at the interface towards the intermediate part of the extraction unit.

In a second embodiment the sample rod is parallel to the central axis along its entire length. This is illustrated by FIG. 2.

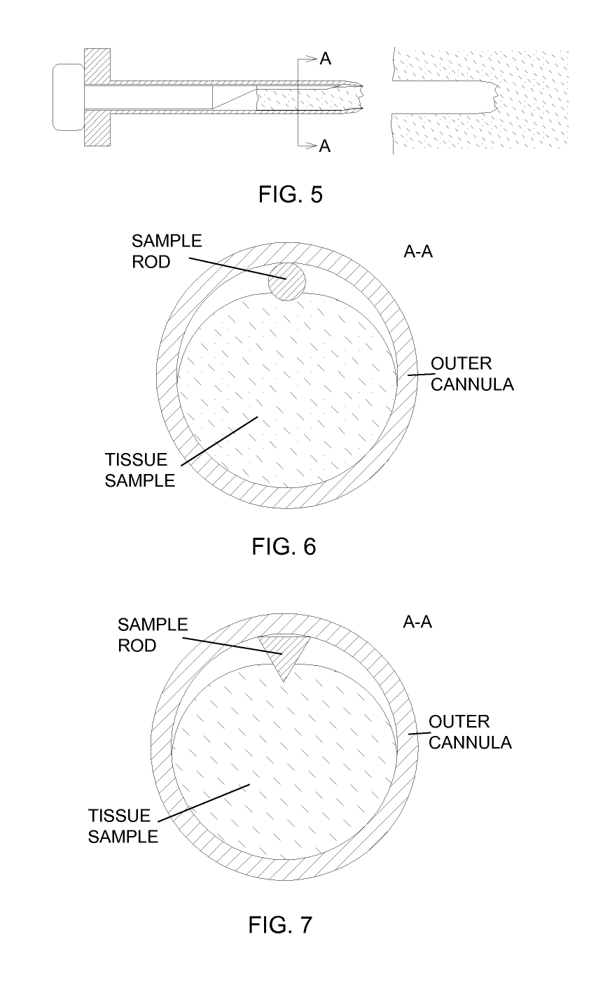

FIG. 6 is a cross-sectional view of A-A in FIG. 5 of the biopsy extraction unit in relation to the biopsy device according to one embodiment.

In this embodiment the sample rod has an essentially circular cross-section.

FIG. 7 is a cross-sectional view of A-A in FIG. 5 of the biopsy extraction unit in relation to the biopsy device according to another embodiment. In this embodiment the sample rod has an essentially triangular cross-section having one side facing outwards in relation to the central axis of the unit.

Further cross-sectional shapes of the sample rod is naturally possible. One important feature is that the rod has a limited surface area in relation to the inner surface area of the outer cannula independent of the cross-sectional shape of the rod. Another important feature is that the rod has a certain width in order to exert radial pressure upon the tissue sample to hold it within the biopsy tube. This width may be in the interval of 3-15% of the surface area, where 15% is a sample rod that covers 15% of the inner surface; in that case the sample rod has a limited thickness. The sample rod illustrated in FIG. 6 covers approximately 5% of the inner surface.

The maximal cross-sectional extension of the sample rod is naturally related to the outer diameter of the outer cannula and to the material thickness of the cannula wall. Therefore, within the scope defined by the claims, the sample rod may have e.g. elliptical, rectangular, quadratic cross-sections.

As an example, the maximal extension is in the range of 100-300% of the thickness of the cannula wall and 10-20% of the outer diameter of the cannula.

Preferably, at least the sample rod is made from metal, e.g. stainless steel or any suitable alloy.

Alternatively, at least the sample rod is made from plastic. The entire biopsy extraction may be made from plastic and would then be a disposable article.

FIGS. 3-5 are cross-sectional side views illustrating the use of the biopsy extraction unit in relation to a biopsy device. A device handle is attached to the outer cannula.

Initially, the outer cannula is inserted into the tissue where a tissue sample is to be taken, this is illustrated in FIG. 3. During the insertion an obturator (not shown) may be arranged within the cannula in order to prevent tissue form entering the biopsy tube prior the tissue sample location is reached.

In FIG. 4 is illustrated the state when the biopsy extraction unit is fully inserted into the outer cannula. It is important that the biopsy extraction unit is used with a mating outer cannula such that the length of the sample rod is such that when the biopsy extraction unit is fully inserted the distal tip of the sample rod is located within the outer cannula. In the figure, L designates the distance between the respective distal tips. L is naturally related to the dimension of the biopsy cannula and may preferably be in the interval of 0-3 mm.

In FIG. 5 the biopsy device including the outer cannula, the biopsy extraction unit and the sample is withdrawn.

Afterwards the tissue sample may be expelled from the biopsy tube in a well-known manner. E.g. by using a suitable rod.

Thus, the method of biopsy tissue sampling, comprises the following steps:
inserting an outer cannula into tissue where a tissue sample is to be taken,
inserting a sample rod of a biopsy extraction unit, as described above, into the outer cannula, and
withdrawing the outer cannula with the sample rod.

The present invention is not limited to the above-described preferred embodiments.

Various alternatives, modifications and equivalents may be used. Therefore, the above embodiments should not be taken as limiting the scope of the invention, which is defined by the appending claims.

1. Biopsy extraction unit for use in relation to a biopsy device provided with an outer cannula,
characterized in that the biopsy extraction unit is provided with a unit handle, an intermediate part and an elongated sample rod, the rod being offset and essentially parallel to a longitudinal central axis of the biopsy extraction unit, wherein the sample rod is adapted to essentially be positioned along an inner wall of the outer cannula when inserted into said biopsy device.

2. Biopsy extraction unit according to claim 1, wherein the sample rod has an essentially circular cross-section.

3. Biopsy extraction unit according to claim 1, wherein the sample rod has an essentially triangular cross-section having one side facing outwards in relation to the central axis of the unit.

4. Biopsy extraction unit according to claim 1, wherein the sample rod has an essentially rectangular cross-section having one side facing outwards in relation to the central axis of the unit.

5. Biopsy extraction unit according to claim 1, wherein the distal tip of the sample rod is sharpened such that the sharpened edge is located furthest away from the central axis in the radial direction.

6. Biopsy extraction unit according to claim 1, wherein the sample rod is parallel to the central axis along its entire length.

7. Biopsy extraction unit according to claim 1, wherein the distal part of the sample rod is bent outwards.

8. Biopsy extraction unit according to claim 1, wherein at least the sample rod is made from metal.

9. Biopsy extraction unit according to claim 1, wherein at least the sample rod is made from plastic.

10. Biopsy device comprising a biopsy extraction unit according to claim 1, wherein the length of the sample rod is such that when the biopsy extraction unit is fully inserted the distal tip of the sample rod is located within the outer cannula.

11. Method of biopsy tissue sampling, comprising:
inserting an outer cannula into tissue where a tissue sample is to be taken,
inserting a sample rod of a biopsy extraction unit according to claim 1, into the outer cannula,
withdrawing the outer cannula and the sample rod, where the cannula encloses a tissue sample.
